(19) United States
(12) Patent Application Publication
(10) Pub. No.: US 2007/0016166 A1
(43) Pub. Date: Jan. 18, 2007

(54) Catheter attachment collet for a central port

(76) Inventor: Robert C. Thistle, Bridgewater, MA (US)

Correspondence Address: FAY KAPLUN & MARCIN, LLP, 15O BROADWAY, SUITE 702, NEW YORK, NY, 10038, US

(21) Appl. No.: 11/177,622

(22) Filed: Jul. 8, 2005

(51) Int. Cl. A61M 39/10

(52) U.S. Cl. 604/533

(57) ABSTRACT

A mechanism for locking a catheter to a port comprises an elongate body defining a flow passage, a port end of the elongate body adapted for attachment to a port, a collet end of the elongate body opposite the port end being biased toward an open configuration in which an in inner diameter of the collet end is sized to receive therein an end of a catheter to be coupled to the port and a tightening device mounted to the elongate body for movement relative thereto between a first position in which the collet end is released to the open configuration and a second position in which the collet end is constricted to a closed configuration in which the inner diameter of the collet end grips the end of the catheter to retain the end of the catheter therewithin.

### BACKGROUND OF THE INVENTION

Catheters are often used to form semi-permanent paths into the body so that fluids may be introduced to and removed from the body without repeatedly inserting needles or tubes. For example, a central port catheter is generally used to access the vascular system with a subcutaneous portion of the catheters tunneled from a location of the central port to a remote vessel access location. Thus, the location of the central port may be selected to maximize ease of access, and patient comfort, etc., while the target vessel is accessed at a desired remote location.

The catheter is generally connected to the central port by inserting a barb extending from the port into a lumen of the catheter with a twist lock being tightened to the port housing to secure the port thereto. However, tightening of the lock adds a step to the procedure and may rotate the catheter around the barb, twisting the catheter body. This twisting may be transmitted to the distal end of the catheter, where it may move the distal end out of a desired positioning within the target vessel.

### SUMMARY OF THE INVENTION

In one aspect, the present invention is directed to a mechanism for locking a catheter to a port, comprising a elongate body defining a flow passage, a port end of the elongate body adapted for attachment to a port, a collet end of the elongate body opposite the port end being biased toward an open configuration in which an in inner diameter of the collet end is sized to receive therein an end of a catheter to be coupled to the port and a tightening device mounted to the elongate body for movement relative thereto between a first position in which the collet end is released to the open configuration and a second position in which the collet end is constricted to a closed configuration in which the inner diameter of the collet end grips the end of the catheter to retain the end of the catheter therewithin.

### BRIEF DESCRIPTION OF THE DRAWINGS

FIG. 1 is a perspective view showing a catheter attachment collet lock according to an embodiment of the present invention; and

FIG. 2 is a cross-sectional perspective view of a catheter attachment collet lock according to a further embodiment of the invention.

### DETAILED DESCRIPTION

The present invention may be further understood with reference to the following description and the appended drawings, wherein like elements are referred to with the same reference numerals. The invention is related to medical devices used to withdraw fluids from and/or infuse fluids into the body. More specifically, the invention is related to a device for securely connecting a catheter to a central port.

As described above, a catheter may be used in conjunction with a central port to facilitate repeated access to blood vessels. Regardless of its use, the central port is designed to be repeatedly connected to and disconnected from an external catheter, which in turn may be connected to an external medical device.

The connection between a central port and a catheter is conventionally formed by pushing an open end of the catheter over a barb extending from the port with a passage in the barb providing fluid communication between a lumen of the catheter and a flow passage of the port.

A catheter attachment apparatus and method according to the present invention eliminates the twisting and displacement common with conventional locks. In addition, the locking device according to the invention eliminates the difficulties inherent in handling the multiple parts that form current twist locks, by providing a lock more integrated with the central port. The attachment procedure is thus simplified as securing the catheter to the outlet of the port does not displace the distal tip and the integrated locking device is easier to handle, since there are no lose parts that may be dropped or misplaced.

The embodiments of the present invention comprise an attachment collet that is fastened to the inlet/outlet of a central port, facing the catheter. The attachment collet is designed to receive one end of the catheter and to compress it, thereby mechanically retaining it therewithin. For example, the catheter may be retained against the collet by friction. A barb may extend from the port along the centerline of the attachment collet, to receive the end of the catheter. The attachment collet, in that case, compresses the catheter between an inner surface of the collet and an outer surface of the barb, to mechanically retain the catheter in place so that fluids may flow from a lumen of the catheter through a passage in the barb to the port.

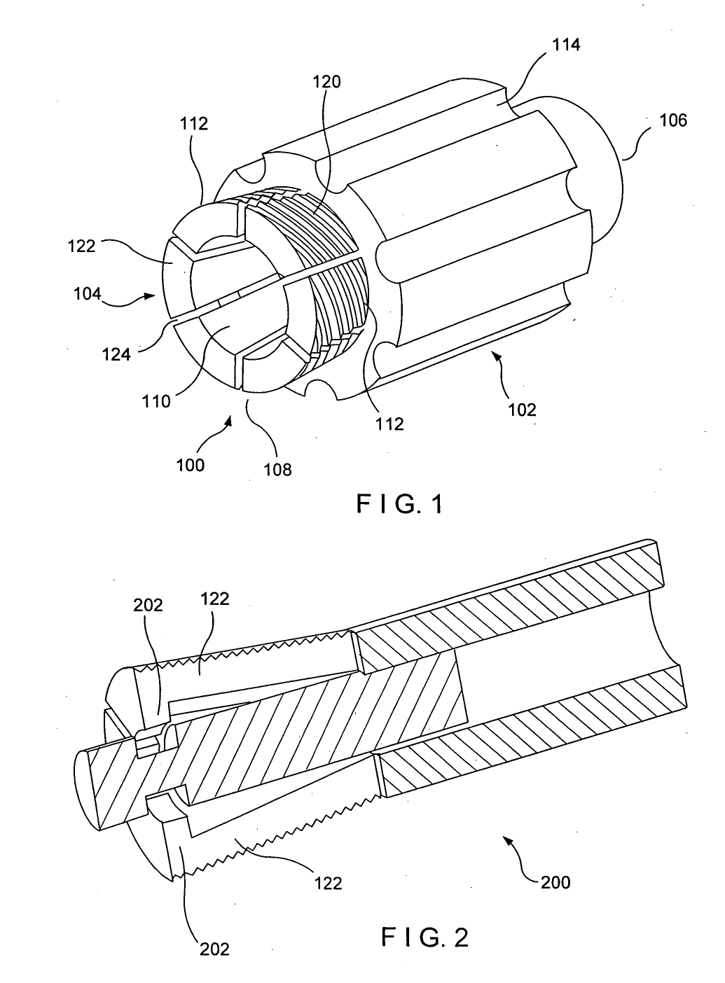

FIG. 1 shows an exemplary embodiment of a catheter attachment collet lock 100 for a central port according to the present invention. The collet lock 100 comprises two principal portions: a collet 104 and a thumb wheel 102. The collet 104 is formed of an elongated tubular body having a port end 106 that can be secured to an inlet or outlet opening of the central port by conventional means such as, for example, an adhesive or mechanical connection. An internal lumen of the collet 104 has a minimum inner diameter sufficient to receive the barb, while providing sufficient flow area to convey a fluid therethrough.

An opposite end 108 of the collet 104, which flares outwardly when in an unconstrained open configuration, is formed of a plurality of jaws 122 separated by slots 124. The flared opposite end 108 be squeezed to a closed configuration having a reduced inner diameter by application of an inwardly directed radial force. The jaws 122 are biased toward the increased diameter open configuration. However, when compressed by a radially inward force, the jaws 122 flex inward to abut one another in closed configuration. The opening 110 is designed to receive therein an end of a catheter when in the open configuration. After the catheter end has been inserted therein, the opening 110 is closed down to the reduced diameter, closed configuration by actuating a tightening device to lock the catheter in place on the central port.

In one exemplary embodiment, the flared end 108 of the collet 104 is squeezed by a tightening device such as a thumb wheel 102, in order to reduce the diameter of the opening 110. For example, an external surface of the collet 104 may comprise threads 120 matching corresponding threads on an inner surface of the thumb wheel 102, to form a threaded interface between the two components. The thumb wheel 102 may optionally comprise grooves 114 to facilitate gripping of the device and turning of the thumb wheel 102. With this threaded interface, as the thumb wheel 102 is rotated about the collet 104 in a first direction, it advances towards the flared end 108 of the collet 104, causing the collet 104 to collapse to the reduced diameter configuration. Rotating the thumb wheel 102 about the collet 104 in a second direction moves the thumb wheel 102 away from the flared end 108 allowing the jaws 122 to spring outward to the increased diameter configuration. As the thumb wheel 102 rotates around the collet 104 and locks the catheter to the collet 104 without any relative rotation of the collet 104 and the catheter, the catheter is not twisted by the locking procedure.

Those skilled in the art will understand that the threads 120 and the corresponding threads of the thumb wheel 102 may be right handed or left handed. In the latter case, when the user turns the thumb wheel 102 clockwise relative to the collet 104, the thumb wheel is advanced toward the flared end 108 (i.e., toward the reduced diameter configuration). Using left handed threading for the thumb wheel 102 thus results in a more natural motion to tighten the collet 104 over the catheter, further simplifying the procedure. Furthermore, those skilled in the art will understand that the thumb wheel 102 may be replaced by any conventional apparatus for tightening the collet 104 over the catheter.

A mechanical stop may optionally be incorporated on the collet 104 to prevent the thumb wheel 102 from becoming detached from the collet lock 100. For example, a positive stop 112 may be formed at the lip of the collet 104, to mechanically prevent the thumb wheel 102 from being advanced too far along the collet 104. In that way, the collet lock 100 remains a single piece, securely attached to the central port. The procedure is therefore simplified, since there are no separate parts of the locking mechanism that can fall or be misplaced during attachment of the catheter or after the catheter has been secured in place. According to the invention, once the catheter has been inserted into the collet 104, there are no parts that need to be assembled to secure the catheter to the port.

According to exemplary embodiments of the invention, the inner diameter of the collet 104, both in the increased and reduced diameter configurations, is selected to prevent damage to the catheter. For example, the collapsed inner diameter is preferably chosen to prevent tightening of the collet 104 to a degree that the structural integrity of the catheter is threatened. At the same time, the collapsed inner diameter of the collet 104 must be sufficiently small that, when closed around the catheter, the collet 104 closes down with sufficient force to prevent leaks between the collet 104 and the catheter and to prevent undesired movement of the catheter relative to the port. A port with no inner barb may be connected to a catheter using the device according to the invention when a sufficient contact area and force between the collapsed collet 104 and the catheter is provided. Since it is difficult to slide the catheter over the barb, removing the barb further simplifies forming the connection of the catheter to the central port.

FIG. 2 shows a catheter attachment collet lock 200 according to a further embodiment of the present invention. The collet lock 200 is constructed substantially similarly to the collet lock 100 except for a detent projection 202 extending from the distal end(s) of one or more jaws 122. A profile of each of the detent projections 202 preferably substantially matches a profile of a detent recess 206 formed at a distal end of the barb. Thus, when the jaws 122 are forced radially inward compressing a portion of a catheter received between an inner surface of the jaws 122 and an outer surface of the barb, the detent projection(s) 202 will urge portions of the wall of the catheter into the detent recess to further secure the catheter within the collet lock 200. Those skilled in the art will understand that the collet lock 200 preferably includes a thumb wheel 102 as described above for securing the catheter within the collet lock 200 and for releasing the catheter therefrom.

The present invention has been described with reference to specific embodiments, and more specifically to catheter attachment collet used for a central port. However, other embodiments may be devised that are applicable to other types of catheters and ports. Accordingly, various modifications and changes may be made to the embodiments, without departing from the broadest spirit and scope of the present invention as set forth in the claims that follow. The specification and drawings are accordingly to be regarded in an illustrative rather than restrictive sense.

What is claimed is:

1. A mechanism for locking a catheter to a port, comprising:
an elongate body defining a flow passage, a port end of the elongate body adapted for attachment to a port, a collet end of the elongate body opposite the port end being biased toward an open configuration in which an in inner diameter of the collet end is sized to receive therein an end of a catheter to be coupled to the port; and
a tightening device mounted to the elongate body for movement relative thereto between a first position in which the collet end is released to the open configuration and a second position in which the collet end is constricted to a closed configuration in which the inner diameter of the collet end grips the end of the catheter to retain the end of the catheter therewithin.

2. The locking mechanism according to claim 1, wherein the port end of the elongate body is adapted for at least one of a mechanical and an adhesive bond to the port.

3. The locking mechanism according to claim 1, further comprising a tightening device received around the collet, movement of the tightening device relative to the collet moving the collet between the first and second positions.

4. The locking mechanism according to claim 3, further comprising a threaded interface between the tightening device and the collet, whereby rotation of the tightening device relative to the collet causes a corresponding motion of the tightening device relative to the collet.

5. The locking mechanism according to claim 3, wherein the tightening device includes a thumb wheel threaded on the elongated body.

6. The locking mechanism according to claim 1, wherein in the closed configuration, the collet has an inner diameter sized to securely retain therein an end of a catheter to be coupled to the port.

7. The locking mechanism according to claim 3, further comprising a mechanical stop preventing movement of the tightening device relative to the collet beyond a predetermined stop location.

8. The locking mechanism according to claim 1, wherein, when attached to the port, the flow passage forms a leak resistant lumen fluidly coupled to the port.

9. The locking mechanism according to claim 1, wherein the collet is biased toward the open configuration.

10. A catheter locking system, comprising:
a collet for receiving an end of a catheter;
a port end adapted to be sealingly connected around an opening of a port;
a lumen extending through the collet to the port end; and
a tightening device movably coupled to the collet, motion of the tightening device relative to the collet moving the collet between an open configuration sized for receipt of the catheter end and a closed configuration in which the end of the catheter is gripped therewithin.

11. The catheter locking system according to claim 10, wherein the collet is biased toward the open configuration.

12. The catheter locking system according to claim 10, further comprising a threaded interface between the collet and the tightening device, rotation of the tightening device about the collet moving the tightening device relative to the collet to move the collet between the open and closed configurations.

13. The catheter locking system according to claim 12, wherein the threaded interface comprises left handed threads.

14. The catheter locking system according to claim 10, wherein the collet, the port end and the tightening device form a unitary collet lock.

15. The catheter locking system according to claim 10, wherein the collet includes a mechanical stop preventing removal of the tightening device from the collet.

16. The catheter locking system according to claim 10, wherein, when in the open configuration, an inner diameter of the collet is larger than an outer diameter of a catheter to be received therein.

17. The catheter locking system according to claim 16, wherein, when in the closed configuration, the inner diameter of the collet is less than an outer diameter of a catheter to be received therein.

18. The catheter locking system according to claim 10, wherein a diameter of the lumen is dimensioned to receive a barb extending from a port to be coupled to the catheter locking system.

19. An implantable port comprising:
a port housing including a lumen extending therewithin;
a collet with a first end sealingly coupled around an opening in the port housing, a second end of the collet being biased toward an increased diameter open configuration and being movable to a reduced diameter closed configuration; and
a thumb wheel threaded on the collet, rotation of the thumb wheel about the collet moving the second end of the collet between the open and closed configurations.

20. The central port according to claim 19, wherein, when in the open configuration, an inner diameter of the second end of the collet is larger than an outer diameter of an end of a catheter to be received therein.

21. The central port according to claim 20, wherein, when in the closed configuration, the inner diameter of the second end of the collet is less than an outer diameter of the end of a catheter to be received therein.

22. The central port according to claim 19, wherein the thumb wheel is threaded on the collet with a left handed thread.

23. The central port according to claim 19, further comprising a barb extending from the port housing into a lumen of the collet.
